(19) United States
(12) Patent Application Publication
(10) Pub. No.: US 2018/0303845 A1
(43) Pub. Date: Oct. 25, 2018

(54) USE OF TLR8 AGONISTS TO TREAT CANCER

(71) Applicant: VentiRx Pharmaceuticals, Inc., Seattle, WA (US)

(72) Inventor: Gregory N. DIETSCH, Snohomish, WA (US)

(21) Appl. No.: 15/771,662

(22) PCT Filed: Nov. 2, 2016
(86) PCT No.: PCT/US2016/060098

§ 371 Date: Apr. 27, 2018

Related U.S. Application Data
(60) Provisional application No. 62/249,878, filed on Nov. 2, 2015.

(51) Int. Cl. A61K 31/55; A61K 39/395; A61K 45/06; C07K 16/28; A61K 31/704; A61P 35/00; C07K 16/30

(52) U.S. Cl. CPC A61K 31/55; A61K 39/3955; A61K 45/06; A61K 2039/505; A61K 31/704; A61P 35/00; C07K 16/30; C07K 16/2863

(57) ABSTRACT

The application relates to methods of inducing antitumor immune responses in a subject having cancer. In some embodiments, methods are provided for administering to a subject in need thereof a TLR8 agonist and a PD1-1/PD-L1 antagonist, optionally including one or more additional therapeutic agents.

### CROSS-REFERENCE TO RELATED APPLICATION

This application claims priority to and the benefit of U.S. provisional application No. 62/249,878, filed Nov. 2, 2015, the contents of which are incorporated herein by reference in its entirety.

### FIELD OF THE DISCLOSURE

Embodiments of the present disclosure are directed to methods of enhancing the cytotoxicity of therapeutic monoclonal antibodies and other therapeutic agents for the treatment of cancer and other cellular diseases.

### BACKGROUND OF THE DISCLOSURE

Despite well-characterized pathways documenting the capacity of the immune system to recognize and destroy malignant cells, cancer remains one of the leading causes of death in developing countries. The development and propagation of adaptive immune responses to tumor-expressed antigens is a highly regulated process. The initial recognition of tumor-expressed antigens in the context of major histocompatibility complex (MHC) and specific costimulatory signals initiates T cell activation, drives clonal expansion and upregulates tumoricidal effector pathways. Subsequent expression of regulatory molecules by these activated T cells, which act as “immune checkpoints,” modulates both the magnitude and duration of the anti-tumor response, thus limiting damage to healthy tissue. In some types of cancer, immune checkpoint molecules appear to restrict the effectiveness of tumor directed immune responses, leading to an imbalance that favors tumor survival and growth.

Enhancing tumor directed immune responses by targeting “immune checkpoints” is a new paradigm in immunotherapy. The recent FDA approval of ipilimumab, a CTLA-4-specific monoclonal antibody (mAb) for the treatment of malignant melanoma is encouraging. Clinical trials with mAbs directed at either the Programed Death-1 (PD-1) receptor, expressed by activated T cells or one of its ligands PD-L1, have produced compelling results in multiple oncology indications, and several of these mAbs have progressed into Phase 3 clinical development, and two of these anti-PD-1 antibodies are now approved for certain clinical uses in the United States, limited in some cases for use in combination with ipilimumab or a BRAF inhibitor.

Another area of immunotherapeutic treatment of cancer relates to a family of proteins designated toll-like receptors (TLRs). TLRs recognize pathogen-associated molecular pattern (PAMP) molecules, and expressed broadly on hematopoietic (e.g., mDCs, pDCs, monocytes, or B cells) and non-hematopoietic cells (e.g., epithelial cells) that activate innate responses and facilitate the development of adaptive responses. Recognition of PAMP ligands and other ligands by TLRs triggers the rapid, coordinated production of chemokines, cytokines and other inflammatory mediators that leads to the generation of cellular (adaptive) immune responses. There are ten unique TLRs expressed in humans, with TLR1, 2, 4, 5 and 6 being expressed on the cell surface, where they primarily serve to recognize extracellular macromolecular ligands from bacteria and fungi. In contrast, TLR3, 7, 8 and 9 are expressed within the endolysosomal compartmental pathway of various cells, where their function is to recognize foreign nucleic acids from intracellular pathogens. It is the endosome-localized TLRs, particularly TLR7, 8 and 9, that have recently emerged as important targets for anticancer immunotherapies. The engagement of specific TLRs leads to the activation of different cell populations and the production of distinct patterns of cytokines and other inflammatory mediators, resulting in alternative immune response profiles. Thus, TLRs are an important target for immunotherapy development because of their central role in inducing and modulating immune responses.

Toll-like receptor-8 (TLR8, also designated CD288; www at ncbi.nlm.nih (dot) gov/gene/51311) was first described in 2000, and was found to be an important element in the innate immune response to viral infection. Since then, stimulation of innate immune mechanisms through TLR8 agonism has also been reported to antagonize tumor growth and proliferation. TLR8 agonists stimulate myeloid-derived dendritic cells (mDC) by binding to the internal TLR8 receptor. This triggers the mDC to produce and release inflammatory mediators (cytokines and chemokines) that activate both the innate and adaptive immune responses.

Motolimod (formerly VTX-2337, VTX-378) is a selective TLR8 agonist that is currently in Phase 2 clinical development for multiple oncology indications. Activation of TLR8 on monocytes and myeloid dendritic cells by motolimod causes not only release of inflammatory mediators, but also certain other changes in their behavior, for example, enhancement of antigen processing and presentation, induction of the expression of costimulatory molecules required for T cell activation, and triggering of the migration of antigen-presenting cells to lymphatic tissues. For example, motolimod has been shown to stimulate both mDCs and monocytes to produce Th1-polarizing cytokines (including IL-12, IFNγ and TNFα), enhance NK-mediated lysis of tumor cells, and augment the development of tumor-specific cytotoxic lymphocytes in murine tumor models. Motolimod also appears to work synergistically with certain types of cancer therapies to stimulate an immune response in the tumor. For example, motolimod also enhances the anti-tumor effect of monoclonal antibodies by stimulating natural killer (NK) cells and antibody-dependent cellular cytotoxicity (ADCC). Motolimod has also been shown to clearly increase the activity of pegylated liposomal doxorubicin in an experimental model of ovarian cancer. This enhancement is associated with the production of various inflammatory mediators, and has been shown to help prime the adaptive T-cell response to the ovarian cancer.

A further need exists to improve the therapeutic effectiveness of immunotherapeutic methods for treatment of cancer. In view of the great therapeutic potential for compounds that modulate TLR and despite the work that has already been done, there is a substantial ongoing need to expand their use and therapeutic benefits. There is a need to enhance effector function of antibodies, for example enhancing the ADCC and/or complement-dependent cytotoxicity (CDC) function of antibodies, and to modulate other suppressive immune functions.

Throughout this description, including the foregoing description of related art, all publicly available documents described herein, including any and all U.S. patents, are specifically incorporated by reference herein in their entirety. The foregoing description of related art is not intended in any way as an admission that any of the documents described therein, including pending United States patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

### SUMMARY OF THE DISCLOSURE

The present disclosure relates to the use of combinations comprising TLR8 agonists and PD-1/PDL1 antagonists in the treatment of cellular diseases such as cancer and immune cell-mediated diseases or disorders. In some embodiments, the combinations comprise a TLR8 agonist, a PD-1/PD-L1 antagonist, and a therapeutic agent, for use in the treatment of cellular diseases such as cancer and immune cell-mediated diseases or disorders.

In one aspect, disclosed herein is a method of treating cancer in a subject in need thereof, comprising administering to said subject a therapeutically effective combination comprising a TLR8 agonist and a PD-1 antagonist.

In another aspect, disclosed herein is a pharmaceutical composition for use in a combinational therapy of treating cancer comprising a TLR8 agonist and a pharmaceutically acceptable carrier, wherein the combinational therapy further comprises administering an effective amount of a PD-1 antagonist.

In some embodiments, the TLR8 agonist is selected from compounds of Formula I:

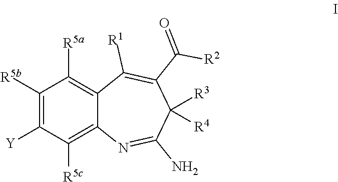

in which,

Y is CF₂CF₃, CF₂CF₂R⁶, or an aryl or heteroaryl ring, wherein said aryl and heteroaryl rings are substituted with one or more groups independently selected from alkenyl, alkynyl, Br, CN, OH, NR⁶R⁷, C(═O)R⁸, NR⁶SO₂R⁷, (C₁-C₆ alkyl)amino, R⁶OC(═O)CH═CH₂—, SR⁶ and SO₂R⁶, and wherein said aryl and heteroaryl rings are optionally further substituted with one or more groups independently selected from F, Cl, CF₃, CF₃O—, HCF₂O—, alkyl, heteroalkyl, and ArO—;

R¹, R³ and R⁴ are independently selected from H, C₁-C₆ alkyl, C₂-C₆ alkenyl, C₂-C₆ alkynyl, C₁-C₆ heteroalkyl, C₃-C₆ cycloalkyl, C₃-C₆ cycloalkenyl, heterocycloalkyl with 3 to 8 ring atoms wherein one atom is selected from nitrogen, oxygen and sulfur, aryl and 5-7 membered heteroaryl, wherein said alkyl, alkenyl, alkynyl, heteroalkyl, cycloalkyl, cycloalkenyl, heterocycloalkyl, aryl and heteroaryl are optionally substituted with one or more substituents independently selected from C₁-C₆ alkyl, C₂-C₆ alkenyl, C₂-C₆ alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH—, NR⁶SO₂R⁷, SR⁶, and SO₂R⁶;

or R³ and R⁴ together with the atom to which they are attached form a saturated or partially unsaturated C₃-C₆ carbocyclic ring, wherein said carbocyclic ring is optionally substituted with one or more substituents independently selected from C₁-C₆ alkyl, C₂-C₆ alkenyl, C₂-C₆ alkynyl, F, Cl, Br, I, CN, OR⁶, NR⁶R⁷, C(═O)R⁶, C(═O)OR⁶, OC(═O)R⁶, C(═O) NR⁶R⁷, (C₁-C₆ alkyl)amino, CH₃OCH₂O—, R⁶OC(═O)CH═CH—, NR⁶SO₂R⁷, SR⁶, and SO₂R⁶;

R² and R⁸ are independently selected from H, OR⁶, NR⁶R⁷, C₁-C₆ alkyl, C₂-C₆ alkenyl, C₂-C₆ alkynyl, C₁-C₆ heteroalkyl, C₃-C₆ cycloalkyl, C₃-C₆ cycloalkenyl, heterocycloalkyl with 3 to 8 ring atoms wherein one atom is selected from nitrogen, oxygen and sulfur, aryl and 5-7 membered heteroaryl, wherein said alkyl, alkenyl, alkynyl, heteroalkyl, cycloalkyl, cycloalkenyl, heterocycloalkyl, aryl and heteroaryl are optionally substituted with one or more substituents independently selected from a C₁-C₆ alkyl, C₂-C₆ alkenyl, C₂-C₆ alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH—, NR⁶SO₂R⁷, SR⁶, and SO₂R⁶;

R⁵a, R⁵b, and R⁵c are independently selected from H, F, Cl, Br, I, OMe, CH₃, CH₂F, CHF₂ and CF₃ and

R⁶ and R⁷ are independently selected from H, C₁-C₆ alkyl, C₂-C₆ alkenyl, C₂-C₆ alkynyl, C₁-C₆ heteroalkyl, C₃-C₆ cycloalkyl, C₃-C₆ cycloalkenyl, heterocycloalkyl with 3 to 8 ring atoms wherein one atom is selected from nitrogen, oxygen and sulfur, aryl and 5-7 membered heteroaryl, wherein said alkyl, alkenyl, alkynyl, heteroalkyl, cycloalkyl, cycloalkenyl, heterocycloalkyl, aryl and heteroaryl are optionally substituted with one or more substituents independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶, and SO₂R⁶,

or R⁶ and R⁷ together with the atom to which they are attached form a saturated or partially unsaturated heterocyclic ring, wherein said heterocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH—, NR⁶SO₂R⁷, SR⁶, and SO₂R⁶; or a metabolite, solvate, tautomer, or pharmaceutically acceptable salt thereof.

In some embodiments, the TLR8 agonist has the structure:

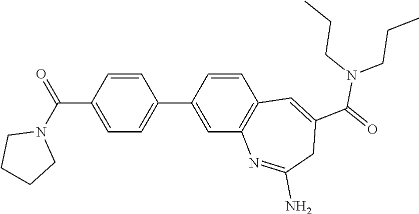 [SMILES: CCCN(CCC)C(=O)C1=Cc2ccc(-c3ccc(C(=O)N4CCCC4)cc3)cc2N=C(N)C1]

In some embodiments, the PD-1 antagonist interferes with binding of PD-1 to PD-L1 or PD-L2. Thus in some embodiments the PD-1 antagonist interferes with a biological activity of PD-1. In some embodiments, the PD-1 antagonist is an antibody that binds to PD-1, such as nivolumab, pembrolizumab, and pidilizumab. In some embodiments, the PD-1 antagonist is an antibody that binds to PD-L1, such as atezolizumab, avelumab, BMS 936559, and durvalumab. In some embodiments, the PD-1 antibody engages the innate immune system or induces ADCC activity.

In some embodiments, the combination further comprises an epidermal growth factor receptor (EGFR) antagonist. In some embodiments, the EGFR antagonist is an anti-EGFR antibody, such as cetuximab, necitumumab, panitumumab, and zalutumumab.

In another aspect, disclosed herein is a method of treating cancer in a subject in need thereof, comprising administering to said subject a therapeutically effective combination comprising a TLR8 agonist, a PD-1 antagonist, and an EGFR antagonist. In some embodiments, the EGFR antagonist is an anti-EGFR antibody such as cetuximab, necitumumab, panitumumab, and zalutumumab.

In another aspect, disclosed herein is a method of treating cancer in a subject in need thereof, comprising administering to said subject a therapeutically effective combination comprising a TLR8 agonist, a PD-1 antagonist, and an anthracycline anticancer agent. In some embodiments, the anticancer anthracycline agent is pegylated liposomal doxorubicin.

In another aspect, disclosed herein is a method of treating cancer in a subject in need thereof, comprising administering to said subject a therapeutically effective combination comprising motolimod, nivolumab, and cetuximab.

In another aspect, disclosed herein is a method of treating cancer in a subject in need thereof, comprising administering to said subject a therapeutically effective combination comprising motolimod, nivolumab, and pegylated liposomal doxorubicin.

In another aspect, in methods of treating cancer in a patient that comprise administering to the patient (a) an anti-EGFR antibody and (b) a TLR8 agonist, disclosed herein is an improvement consisting of administering to the patient (c) a PD-1 antagonist in an amount sufficient to inhibit PD-1 activity in the patient.

In another aspect, disclosed herein is a pharmaceutical composition for use in a combinational therapy of treating cancer comprising a TLR8 agonist and a pharmaceutically acceptable carrier, wherein the combinational therapy further comprises administering an effective amount of a PD-1 antagonist and administering an effective amount of an EGFR antagonist.

In another aspect, disclosed herein is a pharmaceutical composition for use in a combinational therapy of treating cancer comprising a TLR8 agonist and a pharmaceutically acceptable carrier, wherein the combinational therapy further comprises administering an effective amount of a PD-1 antagonist and administering an effective amount of an anthracycline anticancer agent.

In another aspect, disclosed herein is a pharmaceutical composition for use in a combinational therapy of treating cancer comprising motolimod and a pharmaceutically acceptable carrier, wherein the combinational therapy further comprises administering an effective amount of nivolumab and administering an effective amount of cetuximab.

In another aspect, disclosed herein is a pharmaceutical composition for use in a combinational therapy of treating cancer comprising motolimod and a pharmaceutically acceptable carrier, wherein the combinational therapy further comprises administering an effective amount of nivolumab and administering an effective amount of pegylated liposomal doxorubicin.

In another aspect, disclosed herein is a pharmaceutical composition for use in improvement of a combinational therapy of treating cancer comprising (a) an anti-EGFR antibody, wherein the combinational therapy further comprises administering an effective amount of (b) a TLR8 agonist, and the improvement consists of administration of (c) a PD-1 antagonist in an amount sufficient to inhibit PD-1 activity.

### DETAILED DESCRIPTION OF THE DISCLOSURE

The effectiveness of anti-PD1 mAbs in some cancer types demonstrates that the adaptive immune response is recognizing and responding to tumor-expressed antigens. It is possible that PD-1 antagonism acts in part to reactivate previously sensitized T cells. According to the present disclosure, parallel activation of the innate immune system with a TLR8 agonist should potentiate the activity of PD-1 antagonism by increasing the presentation of tumor-expressed antigens to responsive T cells and providing cytokine signals that promote expansion of T cells and reinforce the activity of a tumor-directed cytolytic T cell response. It is believed that these immune-activating features of TLR8 agonists will complement the therapeutic activity of PD-1 antagonists (e.g., anti-PD-1 antibodies) in some cancer types and perhaps open up additional oncology indications where PD-1 antagonists alone have limited activity. In particular, it is believed that in cases where a TLR8 response to a TLR8 agonist is well characterized, a dose can be identified that augments development and propagation of tumor-directed T cell response facilitated by PD-1 antagonists, without over-activating the adaptive response and producing levels of cytokines/chemokines that lead to tolerability/safety concerns.

In one aspect, disclosed herein is a method of treating cancer in a subject in need thereof, comprising administering to said subject a therapeutically effective combination comprising a TLR8 agonist and a PD-1 antagonist.

In some embodiments, the combination further comprises an epidermal growth factor receptor EGFR antagonist. In some embodiments, the EGFR antagonist is an anti-EGFR antibody, such as cetuximab, necitumumab, panitumumab, and zalutumumab.

In another aspect, disclosed herein is a pharmaceutical composition for use in a combinational therapy of treating cancer comprising a TLR8 agonist and a pharmaceutically acceptable carrier, wherein the combinational therapy further comprises administering an effective amount of a PD-1 antagonist.

In another aspect, disclosed herein is a method of treating cancer in a subject in need thereof, comprising administering to said subject a therapeutically effective combination comprising a TLR8 agonist, a PD-1 antagonist, and an EGFR antagonist. In some embodiments, the EGFR antagonist is and anti-EGFR antibody such as cetuximab, necitumumab, panitumumab, and zalutumumab.

In another aspect, disclosed herein is a method of treating cancer in a subject in need thereof, comprising administering to said subject a therapeutically effective combination comprising a TLR8 agonist, a PD-1 antagonist, and an anthracycline anticancer agent. In some embodiments, the anticancer anthracycline agent is pegylated liposomal doxorubicin.

In another aspect, disclosed herein is a method of treating cancer in a subject in need thereof, comprising administering to said subject a therapeutically effective combination comprising motolimod, nivolumab, and cetuximab.

In another aspect, disclosed herein is a method of treating cancer in a subject in need thereof, comprising administering to said subject a therapeutically effective combination comprising motolimod, nivolumab, and pegylated liposomal doxorubicin.

In another aspect, in methods of treating cancer in a patient that comprise administering to the patient (a) an anti-EGFR antibody and (b) a TLR8 agonist, disclosed herein is an improvement consisting of administering to the patient (c) a PD-1 antagonist in an amount sufficient to inhibit PD-1 activity in the patient.

In another aspect, disclosed herein is a pharmaceutical composition for use in a combinational therapy of treating cancer comprising a TLR8 agonist and a pharmaceutically acceptable carrier, wherein the combinational therapy further comprises administering an effective amount of a PD-1 antagonist and administering an effective amount of an EGFR antagonist.

In another aspect, disclosed herein is a pharmaceutical composition for use in a combinational therapy of treating cancer comprising a TLR8 agonist and a pharmaceutically acceptable carrier, wherein the combinational therapy further comprises administering an effective amount of a PD-1 antagonist and administering an effective amount of an anthracycline anticancer agent.

In another aspect, disclosed herein is a pharmaceutical composition for use in a combinational therapy of treating cancer comprising motolimod and a pharmaceutically acceptable carrier, wherein the combinational therapy further comprises administering an effective amount of nivolumab and administering an effective amount of cetuximab.

In another aspect, disclosed herein is a pharmaceutical composition for use in a combinational therapy of treating cancer comprising motolimod and a pharmaceutically acceptable carrier, wherein the combinational therapy further comprises administering an effective amount of nivolumab and administering an effective amount of pegylated liposomal doxorubicin.

In another aspect, disclosed herein is a pharmaceutical composition for use in improvement of a combinational therapy of treating cancer comprising (a) an anti-EGFR antibody, wherein the combinational therapy further comprises administering an effective amount of (b) a TLR8 agonist, and the improvement consists of administration of (c) a PD-1 antagonist in an amount sufficient to inhibit PD-1 activity.

The combination therapy of the disclosure can also be carried out in combination with one or more additional treatment modalities (e.g., radiation therapy, surgery) in a regimen for the treatment of cancer. TLR8 Agonists

In some embodiments, the TLR8 agonist is selected from among those disclosed in international patent applications WO 2007/024612 A2 and WO 2007/040840 A2, the contents of each of which are incorporated by reference in their entireties. Other TLR8 agonists can be identified using methods known in the art for assessing TLR8 agonism in vitro or in vivo.

In some embodiments, the disclosure provides methods of treating cellular diseases such as cancer and immune cell-mediated diseases or disorders in which the TLR8 agonist is of Formula I:

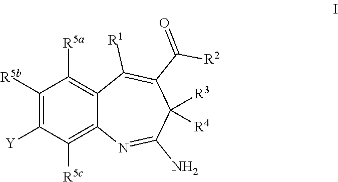

in which,

Y is CF₂CF₃, CF₂CF₂R⁶, or an aryl or heteroaryl ring, wherein said aryl and heteroaryl rings are substituted with one or more groups independently selected from alkenyl, alkynyl, Br, CN, OH, NR⁶R⁷, C(═O)R⁸, NR⁶SO₂R⁷, (C₁-C alkyl)amino, R⁶OC(═O)CH═CH₂—, SR⁶ and SO₂R⁶, and wherein said aryl and heteroaryl rings are optionally further substituted with one or more groups independently selected from F, Cl, CF₃, CF₃O—, HCF₂O—, alkyl, heteroalkyl, and ArO—;

R¹, R³ and R⁴ are independently selected from H, C₁-C₆ alkyl, C₂-C₆ alkenyl, C₂-C₆ alkynyl, C₁-C₆ heteroalkyl, C₃-C₆ cycloalkyl, C₃-C₆ cycloalkenyl, heterocycloalkyl with 3 to 8 ring atoms wherein one atom is selected from nitrogen, oxygen and sulfur, aryl and 5-7 membered heteroaryl, wherein said alkyl, alkenyl, alkynyl, heteroalkyl, cycloalkyl, cycloalkenyl, heterocycloalkyl, aryl and heteroaryl are optionally substituted with one or more substituents independently selected from C₁-C₆ alkyl, C₂-C₆ alkenyl, C₂-C₆ alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH—, NR⁶SO₂R⁷, SR⁶, and SO₂R⁶;

or R³ and R⁴ together with the atom to which they are attached form a saturated or partially unsaturated C₃-C₆ carbocyclic ring, wherein said carbocyclic ring is optionally substituted with one or more substituents independently selected from C₁-C₆ alkyl, C₂-C₆ alkenyl, C₂-C₆ alkynyl, F, Cl, Br, I, CN, OR⁶, NR⁶R⁷, C(═O)R⁶, C(═O)OR⁶, OC(═O)R⁶, C(═O) NR⁶R⁷, (C₁-C₆ alkyl)amino, CH₃OCH₂O—, R⁶OC(═O)CH═CH—, NR⁶SO₂R⁷, SR⁶, and SO₂R⁶;

R² and R⁸ are independently selected from H, OR⁶, NR⁶R⁷, C₁-C₆ alkyl, C₂-C₆ alkenyl, C₂-C₆ alkynyl, C₁-C₆ heteroalkyl, C₃-C₆ cycloalkyl, C₃-C₆ cycloalkenyl, heterocycloalkyl with 3 to 8 ring atoms wherein one atom is selected from nitrogen, oxygen and sulfur, aryl and 5-7 membered heteroaryl, wherein said alkyl, alkenyl, alkynyl, heteroalkyl, cycloalkyl, cycloalkenyl, heterocycloalkyl, aryl and heteroaryl are optionally substituted with one or more substituents independently selected from a C₁-C₆ alkyl, C₂-C₆ alkenyl, C₂-C₆ alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH—, NR⁶SO₂R⁷, SR⁶, and SO₂R⁶;

R⁵a, R⁵b, and R⁵c are independently selected from H, F, Cl, Br, I, OMe, CH₃, CH₂F, CHF₂ and CF₃; and

R⁶ and R⁷ are independently selected from H, C₁-C₆ alkyl, C₂-C₆ alkenyl, C₂-C₆ alkynyl, C₁-C₆ heteroalkyl, C₃-C₆ cycloalkyl, C₃-C₆ cycloalkenyl, heterocycloalkyl with 3 to 8 ring atoms wherein one atom is selected from nitrogen, oxygen and sulfur, aryl and 5-7 membered heteroaryl, wherein said alkyl, alkenyl, alkynyl, heteroalkyl, cycloalkyl, cycloalkenyl, heterocycloalkyl, aryl and heteroaryl are optionally substituted with one or more substituents independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶, and SO₂R⁶,

or R⁶ and R⁷ together with the atom to which they are attached form a saturated or partially unsaturated heterocyclic ring, wherein said heterocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH—, NR⁶SO₂R⁷, SR⁶, and SO₂R⁶.

The disclosure also relates to methods in which the TLR8 agonist is a metabolite, solvate, tautomer, or pharmaceutically acceptable salt of a compound of Formula I.

In some embodiments, the TLR8 agonist is of Formula I as described, in which:

Y is an aryl ring substituted with C(═O)R⁸, and wherein said aryl ring is optionally further substituted with one or more substituents independently selected from F, Cl, CF₃, CF₃O—, HCF₂O—, C₁-C₆ alkyl, C₁-C₆ heteroalkyl and ArO—.

In some embodiments, the TLR8 agonist is of Formula I as described, in which:

R⁶ and R⁷ are independently selected from H, C₁-C₆ alkyl, C₂-C₆ alkenyl, C₂-C₆ alkynyl, C₁-C₆ heteroalkyl, C₃-C₆ cycloalkyl, C₃-C₆ cycloalkenyl, heterocycloalkyl with 3 to 8 ring atoms wherein one atom is selected from nitrogen, oxygen and sulfur, aryl and 5-7 membered heteroaryl, wherein said alkyl, alkenyl, alkynyl, heteroalkyl, cycloalkyl, cycloalkenyl, heterocycloalkyl, aryl and heteroaryl are optionally substituted with one or more substituents independently selected from C₁-C₆ alkyl, C₂-C₆ alkenyl, C₂-C₆ alkynyl, F, Cl, Br, I, CN, O-alkyl, NH₂, —C(═O)alkyl, C(═O)H, C(═O)OH, C(═O)Oalkyl, OC(═O)H, OC(═O)Alkyl, (C₁-C₆ alkyl)amino, (C₁-C₆ alkyl)₂amino, CH₃OCH₂O—, and alkyl-OC(═O)CH═CH—.

In some embodiments, the TLR8 agonist is of Formula I as described, in which:

R⁶ and R⁷ together with the atom to which they are attached form a saturated or partially unsaturated heterocyclic ring with 3 to 8 ring atoms wherein one atom is selected from nitrogen, oxygen and sulfur, wherein said heterocyclic ring is optionally substituted with one or more substituents independently selected from C₁-C₆ alkyl, C₂-C₆ alkenyl, C₂-C₆ alkynyl, F, Cl, Br, I, CN, OR⁶, NH₂, —C(═O)alkyl, C(═O)H, C(═O)OH, C(═O)Oalkyl, OC(═O)H, OC(═O)alkyl, (C₁-C₆ alkyl)amino, (C₁-C₆ alkyl)₂amino, CH₃OCH₂O—, and alkyl-OC(═O)CH═CH—.

In some embodiments, the TLR8 agonist is of Formula I as described, in which R² is OR⁶.

In some embodiments, the TLR8 agonist is of Formula I as described, in which R⁶ is C₁-C₆ alkyl, such as ethyl.

In some embodiments, the TLR8 agonist is of Formula I as described, in which R² is —NR⁶R⁷.

In some embodiments, the TLR8 agonist is of Formula I as described, in which R² is —NR⁶R⁷ and R⁶ and R7 are independently selected from H, C₁-C₆ alkyl and C₁-C₆ heteroalkyl, such as, for example, R⁶ and R⁷ are H, ethyl, propyl or CH₂CH₂OCH₃.

In some embodiments, the TLR8 agonist is of Formula I as described, in which Y is phenyl.

In some embodiments, the TLR8 agonist is of Formula I as described, in which R₈ is selected from OR₆, —NR⁶R⁷, and heterocycloalkyl with 3 to 8 ring atoms wherein one atom is selected from nitrogen, oxygen and sulfur.

In some embodiments, the TLR8 agonist is of Formula I as described, in which R⁸ is heterocycloalkyl with 5 or 6 ring atoms wherein one atom is selected from nitrogen, oxygen and sulfur. For example, R⁸ is pyrrolidine.

In some embodiments, the TLR8 agonist is of Formula I as described, in which R⁶ and R⁷ are independently selected from H and C₁-C₆ alkyl.

In some embodiments, the TLR8 agonist is of Formula I as described, in which Y is

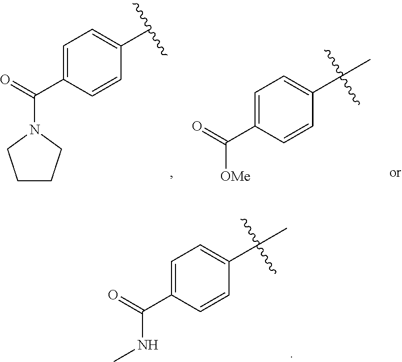 [SMILES: *c1ccc(C(=O)N2CCCC2)cc1 ; *c1ccc(C(=O)OC)cc1 ; *c1ccc(C(=O)NC)cc1]

In some embodiments, the TLR8 agonist is of Formula I as described, in which each of R¹, R³, R⁴, R⁵a, R⁵b, and R⁵c is hydrogen.

For example, the disclosure relates to methods in which the TLR8 agonist is selected from:
(1E,4E)-ethyl-2-amino-8-(4-pyrrolidine-1-carbonyl)phenyl)-3H-benzo[b]azepine-4-carboxylate;
(1E,4E)-ethyl-2-amino-8-(4-(methoxycarbonyl)phenyl)-3H-benzo[b]azepine-4-carboxylate;
(1E,4E)-ethyl2-amino-8-(4-(methylcarbamoyl)phenyl)-3H-benzo[b]azepine-4-carboxylate;
(1E,4E)-2-amino-N,N-dipropyl-8-(4-(pyrrolidine-1-carbonyl)phenyl)-3H-benzo[b]azepine-4-carboxamide and pharmaceutically acceptable salts thereof.

In some embodiments, the TLR8 agonist is motolimod, which is the USAN designation for the compound {2-amino-8-[4-(pyrrolidinylcarbonyl)phenyl]-(3H-benzo[f]azepin-4-yl)}-N,N-dipropylcarboxamide, alternatively 2-amino-N,N-dipropyl-8-[4-(pyrrolidin-1-ylcarbonyl)phenyl]-3H-1-benzazepine-4-carboxamide (formerly known as VTX-2337 or VTX-378), having the chemical structure:

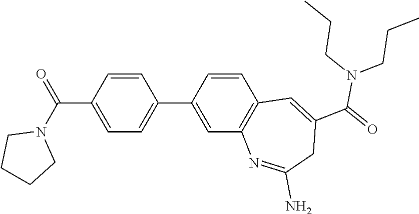 [SMILES: CCCN(CCC)C(=O)C1=Cc2ccc(-c3ccc(C(=O)N4CCCC4)cc3)cc2N=C(N)C1]

The TLR8 agonist is formulated for administration by any route compatible with its physicochemical and pharmaceutical properties. For example, the TLR8 agonist may be formulated for administration by intravenous, intradermal, transdermal, subcutaneous, or intramuscular route. In some embodiments, the TLR8 agonist is formulated for intravenous administration. In some embodiments, the TLR8 agonist is formulated for subcutaneous administration. However, the TLR8 agonist may be formulated for any suitable route of administration, including, by way of example, nasal (e.g., via an aerosol), buccal (e.g., sub-lingual), topical (i.e., administration by either skin and/or mucosal surfaces, including airway surfaces), intrathecal, intra-articular, intrapleural, intracerebral, intra-arterial, intraperitoneal, oral, intralymphatic, intranasal, rectal or vaginal administration, by perfusion through a regional catheter, or by direct intralesional (or intratumoral) injection.

In certain embodiments, the dose of the TLR8 agonist is measured in units of mg/kg of body weight. In other embodiments, the dose is measured in units of mg/kg of lean body weight (i.e., body weight minus body fat content). In other embodiments, the dose is measured in units of mg/m² of body surface area. In other embodiments, the dose is measured in units of mg per dose administered to a patient. Any measurement of dose can be used in conjunction with the compositions and methods of the disclosure and dosage units can be converted by means standard in the art.

To illustrate, in some embodiments, a dose of motolimod can be between 0.1-10 mg/m² (e.g., 0.1-3.9 mg/m², 0.1-1 mg/m², 0.1-2 mg/m², 0.1-4 mg/m², 2-4 mg/m², 2-6 mg/m², 2-8 mg/m²). This includes 0.1 mg/m², 1 mg/m², 2 mg/m², 2.5 mg/m², 3 mg/m², 4 mg/m², 5 mg/m², 6 mg/m², 7 mg/m², 8 mg/m² and points in-between. In some embodiments, the dose of motolimod is about 0.5 mg/m² to about 5 mg/m². In some embodiments, the dose of motolimod is about 2 mg/m² to about 3 mg/m². The frequency of administration is preferably once every 7 to 21 days (e.g., once every 7, 10, 14, 18, 21 days). In some embodiments, the frequency of administration is preferably 1, 2, or 3 times every 7 to 21 days (e.g., once every 7, 10, 14, 18, 21 days). The TLR8 agonist may be given until disease progression or unacceptable toxicity. In some embodiments, 2-20 doses are given (e.g., 2, 4, 6, 8, 10, 12, 14, 16, 18, 20 doses). For example, one mode of administration of motolimod or another TLR8 agonist of Formula I is subcutaneous. In certain further embodiments, motolimod is administered to the subject on a weekly or biweekly basis.

Dosages for intradermal, intramuscular, intraperitoneal, subcutaneous, epidural, or intravenous administration of a TLR8 agonist can be in the range of about 0.02 to 10 mg/kg of body weight, depending on the pharmacokinetic properties of the compound, with routes, amounts, and frequency of dosing being further determined by the nature of the disease being treated and the medical condition of the subject. Suitable doses for topical administration can be in the range of about 0.001 milligram to about 50 milligrams per kilogram of body weight per day, depending on the area of administration. Those skilled in the art will appreciate that dosages are generally higher and/or frequency of administration greater for initial treatment as compared with maintenance regimens.

Examples of dosing regimens that can be used in the methods of the disclosure include, but are not limited to, daily, three times weekly (intermittent), weekly, every 14 days, or every 28 days. In certain embodiments, dosing regimens include, but are not limited to, monthly dosing or dosing every 6-8 weeks. In one embodiment, a TLR8 agonist is administered by subcutaneous injection weekly or biweekly in combination with a suitable PD-1 antagonist for the treatment of cancer or infectious disease in a subject, preferably a human subject. To illustrate, in treating a patient with head and neck cancer, motolimod (3.0 mg/m²) can be administered on days 8 and 15 of a 21-day cycle for 6 cycles, followed by dosing on days 8 and 22 of 28-day cycles until disease progression.

In certain embodiments, the TLR8 agonist is administered prior to, concurrently with, or subsequent to the administration of the one or more therapeutic antibodies. In one embodiment, the TLR8 agonist is formulated with one or more therapeutic antibodies. In another embodiment, the one or more therapeutic antibodies is administered in a separate pharmaceutical composition. In accordance with this embodiment, the one or more therapeutic antibodies may be administered to a subject by the same or different routes of administration as those used to administer TLR8 agonist.

Preferably, motolimod is formulated at a concentration of from about 0.001 mg/mL to about 50 mg/mL, from about 0.01 mg/mL to about 50 mg/mL, from about 0.5 mg/mL to about 50 mg/mL, from about 1 mg/mL to about 40 mg/mL, or from about 2 mg/mL to about 15 mg/mL. In certain embodiments, motolimod is formulated at a concentration of from about 0.5 mg/mL to about 10 mg/mL, from about 0.5 mg/mL to about 8 mg/mL, from about 0.5 mg/mL to about 6 mg/mL, from about 0.5 mg/mL to about 4 mg/mL, or from about 0.5 mg/mL to about 2 mg/mL. In certain embodiments, motolimod is formulated at a concentration of about 0.5 mg/mL, about 1 mg/mL, about 2 mg/mL, about 4 mg/mL, about 6 mg/mL, about 8 mg/mL, about 10 mg/mL, about 15 mg/mL, about 20 mg/mL, about 25 mg/mL, about 30 mg/mL, about 40 mg/mL, or about 50 mg/mL.

Suitable formulations of motolimod are described, for example in international patent publication WO 2007/040840 A2. Such formulations comprise about 1-30%, 5-15%, or 5-10% weight/volume (w/v) of a cyclodextrin, preferably a β-cyclodextrin, and most preferably sulfobutyl ether β-cyclodextrin. In certain embodiments, the formulation comprises 1%, 5%, 10%, 15%, 20%, 25%, or 30% w/v of a cyclodextrin, preferably a β-cyclodextrin, and most preferably sulfobutyl ether β-cyclodextrin. In a particular embodiment, the formulation is an aqueous solution comprising motolimod at a concentration of at least 2 mg/mL. In a further embodiment, the formulation comprises 15% w/v of a cyclodextrin, preferably a β-cyclodextrin, and most preferably sulfobutyl ether β-cyclodextrin. In preferred embodiments, the formulation is suitable for injection in a mammal, preferably a human. In particular embodiments, injection is by a subcutaneous route, an intramuscular route, or transdermal route. In certain embodiments, the formulation is suitable for intravenous administration. In particular embodiments, the formulation is administered by subcutaneous injection.

It is believed that, in the combination therapy methods of the disclosure, the TLR8 agonist enhances or improves the effector activity of antibodies used in the combination therapy, regardless of the antigen-binding activity of the antibodies. Thus, the methods of the disclosure are generally useful for treating or alleviating a symptom of any disorder in which enhanced antibody effector activity is desired in a subject in need thereof. The TLR8 agonist may improve immune status of a patient by potentiating a PD-1 antagonist, such as offsetting the down-regulation of the immune system normally caused by PD-1 preventing the activation of T-cells. The TLR8 agonist may improve ADCC by activating NK cells or CD56⁺ cells either directly or indirectly. Additionally, having a greater proportion of activated NK cells may help overcome the poor ADCC observed in a subset of patients that have low affinity Fc receptors. A TLR8 agonist can also stimulate the secretion of Th1 cytokines, such as TNF-α, IFN-γ, IL-12p40, and IL-12p70 in cells.

### PD-1 Antagonists

The methods of treatment of cancer according to the disclosure include administering to a subject a PD-1 antagonist. “PD-1 antagonist,” as used herein, means any compound (such as an antibody, or other molecule) that interferes with binding between PD-1 and any of its ligands (including, but not limited to PD-L1 and PD-L2) or that inhibits an activity of PD-1 or any of its ligands that is activated by such binding. For example, insofar as PD-1 is implicated in inhibiting the immune response against cancer cells, then, in some embodiments, an effective amount of a PD-1 antagonist is an amount that when administered in the method of the disclosure reduces PD-1 pathway-mediated inhibition of such anti-tumor immune responses. Accordingly, the PD-1 antagonist can be selected to interfere with binding of PD-1 to PD-L1 or PD-L2. Thus, in some cases, the PD-1 antagonist is selected to interfere with a biological activity of PD-1. In some embodiments, the PD-1 antagonist is an antibody that binds to PD-1. In some embodiments, the PD-1 antagonist is an antibody that binds to PD-L1. In some embodiments, the PD-1 antagonist is an antibody that binds to PD-L2.

PD-1 antagonists useful according to the combination therapy methods of the disclosure include, for example, inhibitors of PD-1/PD-L1 interaction, such as atezolizumab, avelumab, BMS 936559, durvalumab, nivolumab, pembrolizumab, and pidilizumab.

In some embodiments, the PD-1 antagonist is an antibody that binds to PD-1, and blocks interaction between PD-1 and one or more of its ligands, PD-L1 and PD-L2. Such anti-PD-1 antibodies include, for example, nivolumab (OPDIVO®; ONO-4538, BMS-936558, or MDX1106), pembrolizumab (KEYTRUDA®; lambrolizumab, MK-3475), and pidilizumab (CT-011).

In some embodiments, the PD-1 antagonist is an antibody that binds to programmed cell death ligand 1 (PD-L1; also designated B7-H1 or CD274), and blocks interaction with its receptor PD-1. Such anti-PD-L1 antibodies include, for example, atezolizumab (MPDL3280A), avelumab (MSB0010718C), BMS-936559, and durvalumab (MEDI4736).

Doses and administration regimens for PD-1 antagonists are generally consistent with those provided for their use when used as single agents. For example, in some embodiments, the dose for nivolumab or pembrolizumab is about 1-10 mg/m². This includes about 1 mg/kg, about 2 mg/kg, about 3 mg/kg, about 4 mg/kg, about 5 mg/kg, about 6 mg/kg, about 7 mg/kg, about 8 mg/kg, about 10 mg/kg and other doses in that range. These anti-PD-1 antibodies are administered by intravenous infusion (e.g., as a 30 min., 45 min., 60 min., 90 min, or 120 min infusion). The frequency of administration may be one time every 7 to 21 days (e.g., once every 10, 14, 18, 21, etc. days). For example, nivolumab may be administered in an amount of 3 mg/kg by intravenous infusion over one hour, every two weeks, continued until disease progression or unacceptable toxicity. As another example, pembrolizumab may be administered in an amount of 2 mg/kg by intravenous infusion over 30 minutes, every three weeks, continued until disease progression or unacceptable toxicity.

Combinations with Therapeutic agents

In another aspect, the therapeutic combination of the disclosure comprises a TLR8 agonist and a PD-1 antagonist in combination with one or more therapeutic agents. A “therapeutic agent” is a chemical compound (e.g., a small molecule, antibody or other protein) useful in the treatment of cancer, regardless of mechanism of action. Therapeutic agents include, but are not limited to, the following groups of compounds: cytotoxic antibiotics, antimetabolites, anti-mitotic agents, alkylating agents, platinum containing compounds, arsenic compounds, DNA topoisomerase inhibitors (topoisomerase I inhibitors and topoisomerase II inhibitors), tyrosine kinase inhibitors, taxanes, nucleoside analogues, plant alkaloids, and toxins, and derivatives thereof.

The following are non-limiting examples of particular compounds within these groups of therapeutic agents. Alkylating agents include nitrogen mustards such as cyclophosphamide, ifosfamide, trofosfamide, and chlorambucil; nitrosoureas such as carmustine and lomustine; alkylsulphonates such as busulfan and treosulfan; and triazenes such as dacarbazine. Platinum-containing compounds include cisplatin, carboplatin, aroplatin, and oxaliplatin. Plant alkaloids include vinca alkaloids such as vincristine, vinblastine, vindesine, and vinorelbine; and taxoids such as paclitaxel (including nanoparticle albumin-bound (nab)-paclitaxel) and docetaxel. DNA topoisomerase inhibitors include epipodophyllotoxins (such as etoposide, teniposide), topotecan, 9-aminocamptothecin, camptothecin, and crisnatol, mitoxantrone; and mitomycins such as mitomycin C. Anti-folates include DHFR inhibitors such as methotrexate and trimetrexate; IMP dehydrogenase inhibitors such as mycophenolic acid, tiazofurin, ribavirin, hydroxyurea and EICAR; and ribonucleotide reductase inhibitors such as deferoxamine. Pyrimidine analogs include uracil analogs such as 5-fluorouracil, floxuridine, doxifluridine, and raltitrexed; and cytosine analogs such as cytarabine (ara-C), cytosine arabinoside, and fludarabine. Purine analogs include mercaptopurine and thioguanine. DNA antimetabolites include 3-HP, 2′-deoxy-5-fluorouridine, 5-HP, alpha-TGDR, aphidicolin glycinate, 5-aza-2′-deoxycytidine, gemcitabine, beta-TGDR, cyclocytidine, guanazole, inosine glycodialdehyde, macebecin II, and pyrazoloimidazole. Antimitotic agents include allocolchicine, halichondrin B, colchicine, colchicine derivative, dolastatin 10, maitansine, rhizoxin, thiocolchicine, and trityl cysteine. Tyrosine kinase inhibitors include imatinib (GLEEVEC®), lapatinib (TYKERB®), and sunitinib (SUTENT®).

Other examples of therapeutic agents for use with the combination therapy of the disclosure include isoprenylation inhibitors; dopaminergic neurotoxins such as 1-methyl-4-phenylpyridinium ion; cell cycle inhibitors such as staurosporine; actinomycins such as actinomycin D and dactinomycin; bleomycins such as bleomycin A2, bleomycin B2, and peplomycin; anthracycline topoisomerase inhibitors such as daunorubicin, doxorubicin, aclarubicin (aclacinomycin A), idarubicin, epirubicin, pirarubicin, zorubicin, and mitoxantrone; MDR inhibitors such as verapamil; and Ca²⁺ ATPase inhibitors such as thapsigargin.

Other examples of suitable therapeutic agents include IFNγ, IL-2, dacarbazine, temozolomide, tamoxifen, carmustine, melphalan, procarbazine, vinblastine, capecitabine, carboplatin, cisplatin, paclitaxel, cyclophosphamide, doxorubicin (ADRIAMYCIN®), rituximab (RITUXAN®), trastuzumab (HERCEPTIN®), imatinib (GLEEVEC®), sunitinib (SUTENT®), gefitinib (IRESSA®), bevacizumab (AVASTIN®), cetuximab (ERBITUX®), or erlotinib (TARCEVA®), an enediyne such as calicheamicin and esperamicin; duocarmycin, methotrexate, doxorubicin, melphalan, chlorambucil, ara-C (cytarabine), vindesine, mitomycin C, cis-platinum, etoposide, bleomycin, and 5-fluorouracil.

In another aspect, the combinations and methods of the disclosure include a therapeutic agent that is a therapeutic antibody. In some embodiments, such antibodies have in vivo therapeutic and/or prophylactic uses against cancer and other cellular diseases.

Typical examples of therapeutic antibodies useful according to the disclosure include, for instance, rituximab (RITUXAN®; MABTHERA®; anti-CD20), cetuximab (ERBITUX®; anti-EGFR), panitumumab (VECTIBIX®; anti-EGFR), trastuzumab (HERCEPTIN®; anti-HER2/neu), alemtuzumab (CAMPATH®, LEMTRADA®; anti-CD52), epratuzumab (anti-CD22), basiliximab (SIMULECT®; anti-CD25), daclizumab (ZENAPAX®; anti-CD25), infliximab (REMICADE®; anti-TNF-α), omalizumab (XOLAIR®; anti-IgE), efalizumab (RAPTIVA®; anti-CD11a), and natalizumab (TYSABRI®; anti-α4-integrin). Such antibodies may be used according to clinical protocols that have been authorized for use in human subjects. A person skilled in the art would recognize that other therapeutic antibodies are useful in the methods of the disclosure.

Within the context of this disclosure, the term “therapeutic antibody” designates more specifically any antibody that functions to deplete target cells in a patient. Specific examples of such target cells include tumor cells, virus-infected cells, allogenic cells, pathological immunocompetent cells (e.g., B lymphocytes, T lymphocytes, antigen-presenting cells, etc.) involved in cancers, allergies, autoimmune diseases, allogenic reactions. Most preferred target cells within the context of this disclosure are tumor cells and virus-infected cells. The therapeutic antibodies may, for instance, mediate a cytotoxic effect or cell lysis, particularly by antibody-dependent cell-mediated cytotoxicity (ADCC).

ADCC requires leukocyte receptors for the Fc portion of IgG (FcγR) whose function is to link the IgG-sensitized antigens to FcγR-bearing cytotoxic cells and to trigger the cell activation machinery. While this mechanism of action has not been evidenced in vivo in humans, it may account for the efficacy of such target cell-depleting therapeutic antibodies. Therefore, the therapeutic antibody is capable of forming an immune complex. For example, an immune complex can be a tumoral target covered by therapeutic antibodies. The therapeutic antibodies may by polyclonal or, preferably, monoclonal. They may be produced by hybridomas or by recombinant cells engineered to express the desired variable and constant domains. The antibodies may be single chain antibodies or other antibody derivatives retaining the antigen specificity and the lower hinge region or a variant thereof. These may be polyfunctional antibodies, recombinant antibodies, humanized antibodies, fragments or variants thereof. Said fragment or a derivative thereof can be selected from a Fab fragment, a Fab′₂ fragment, a CDR, and a ScFv. Therapeutic antibodies are generally specific for surface antigens, e.g., membrane antigens. Most therapeutic antibodies are specific for tumor antigens (e.g., molecules specifically expressed by tumor cells), such as CD20, CD52, ErbB2 (or HER2/Neu), CD33, CD22, CD25, MUC-1, CEA, KDR, αVβ33, particularly lymphoma antigens (e.g., CD20), in some embodiments polyfunctional antibodies may be employed such as an antibody having dual specificity or bispecific antibodies. In some embodiments, therapeutic antibodies have human or non-human primate IgG1 or IgG3 Fc portion. In some embodiments, the therapeutic antibody has a human IgG1 Fc portion.

### Anthracycline Compounds

In one aspect, the combination therapy includes a TLR8 agonist, a PD1-antagonist, and a therapeutic agent that is an anthracycline compound. As used herein, “anthracycline compound” means any of a family of compounds originally obtained from Streptomyces sp., and various synthetic derivative compounds, having antineoplastic properties, e.g., DNA intercalation, topoisomerase II inhibition, and generation of free radicals. For example, anthracycline compounds include daunorubicin (daunomycin), liposomal daunorubicin (e.g., DAUNOXOME®), doxorubicin (e.g., ADRIAMYCIN, RUBEX), aclarubicin, epirubicin, idarubicin, or valrubicin.

In some embodiments the anthracycline compound is doxorubicin, or doxorubicin HCl, or is provided in a non-pegylated liposomal format, (e.g., MYOCET™), or pegylated liposomal format (e.g., DOXIL® or CAELYX™). In some embodiments, the therapeutic agent is pegylated liposomal doxorubicin, such as doxorubicin HCl liposome injection (e.g., DOXIL®).

In some embodiments, the combination therapy includes a dose of doxorubicin of 40 mg/m² to 60 mg/m², administered by intravenous infusion, with subsequent doses given every 21 to 28 days. Alternatively, a dose of doxorubicin is 60 mg/m² to 75 mg/m², administered by intravenous infusion, with subsequent doses given every 21 days. In some embodiments, the dose of pegylated liposomal doxorubicin is 40 mg/m², or 50 mg/m². For pediatric patients, the combination may include a dose of doxorubicin of 35 mg/m² to 75 mg/m² as a single dose repeated every 21 days, or 20 mg/m² to 30 mg/m² once weekly, or 60 mg/m² to 90 mg/m² given as a continuous infusion over 96 hours every 3 to 4 weeks. Doses may be reduced, such as by 25%, 50%, or 75%, in patients with liver disease.

In the case of pegylated liposomal doxorubicin, for example, a dose of 40 mg/m² or 50 mg/m² is administered intravenously at an initial rate of 1 mg/min to minimize the risk of infusion reactions. If no infusion-related reactions occur, the rate of infusion can be increased to complete administration over 1 hour. Subsequent doses of pegylated liposomal doxorubicin may be given at 28-day intervals until disease progression or unacceptable toxicity.

In some embodiments, a combination of the disclosure, optionally including an anthracycline compound, such as pegylated liposomal doxorubicin, is used for the treatment of platinum-resistant ovarian cancer. In some embodiments, a combination of the disclosure, optionally including an anthracycline compound, such as pegylated liposomal doxorubicin, is used for the treatment of platinum-sensitive ovarian cancer or previously untreated disease. In some embodiments, a combination of the disclosure, optionally including an anthracycline compound such as pegylated liposomal doxorubicin, is used for the treatment of recurrent or persistent epithelial ovarian, fallopian tube, or primary peritoneal cancer. The disclosure also provides methods of increasing the effectiveness of an anthracycline compound in the treatment of platinum-resistant ovarian cancer, or recurrent or persistent epithelial ovarian, fallopian tube, or primary peritoneal cancer, through use in a combination therapy that further comprises a TLR8 agonist and a PD-1 antagonist.

### EGFR Antagonists

Head and neck cancer is an immunosuppressive disease, with low absolute lymphocyte counts, impaired activity of effectors such as natural killer (NK) cells, and poor antigen-presenting function. The anti-EGFR monoclonal antibody cetuximab prolongs survival in a subset of patients treated for head and neck cancer; however, there are currently no useful tests to identify patients who will respond to cetuximab therapy, notably because EGFR levels do not correlate with the clinical responses observed. The clinical activity of cetuximab has been mechanistically linked to NK-mediated ADCC. However, recent data has shown that cetuximab increases the frequency of intratumoral CTLA-4⁺FoxP3⁺ regulatory T cells (Treg), which suppress ADCC and other elements of the immune system that can have anti-tumor function, and are associated with poor clinical outcome. In ex vivo experiments, this effect could be attenuated by targeting CTLA-4 on regulatory T lymphocytes (Tregs). Therefore, it is possible that the clinical efficacy of cetuximab may be improved by strategies to intensify the activation of the immune system or inhibit or counteract the Treg-mediated suppressive effects caused by the antibody.

As noted, motolimod is a TLR8 agonist that stimulates myeloid dendritic cells (mDC), monocytes, and NK cells. Preclinical data have demonstrated that motolimod enhances cetuximab and NK-mediated lysis of head and neck cancer cells and dendritic cross-priming of EGFR-specific CD8⁺ T cells. Cetuximab and motolimod in head and neck cancer patients was tolerable and active in a phase 1b study, with increased mobilization and activation of NK cells.

Applicants consider that that NK and monocyte/mDC activation by anti-EGFR antibodies such as cetuximab can be enhanced by the concomitant administration of motolimod, augmenting the innate and adaptive immune response in the circulation and in the tumor microenvironment. Accordingly, in some embodiments of the disclosure, the immunomodulatory effects of cetuximab plus motolimod will be further amplified by the inclusion of PD-1 antagonist, such as nivolumab.

In one aspect, the combination therapy of the disclosure includes effective combinations of a TLR8 agonist, a PD1-antagonist, and a therapeutic agent that is an epidermal growth factor receptor antagonist (EGFR antagonist). EGFR antagonists are compounds that bind to or interact with EGFR or its ligand to prevent normal interactions or activation of EGFR and the biological activities associated with or dependent on this receptor. For example, the EGFR antagonist can be an agent that inhibits or blocks activation of EGFR.

In some embodiments, the EGFR antagonist is a small molecule compound, such as erlotinib (TARCEVA®) or gefitinib (IRESSA®), or a compound with dual EGFR antagonist and HER2/Neu antagonist properties, such as lapatinib (TYKERB®).

In some embodiments, the EGFR antagonist is a larger compound that specifically binds EGFR, such as an anti-EGFR antibody or antibody fragment. In some embodiments, the EGFR antagonist is an anti-EGFR monoclonal antibody. In some embodiments, the EGFR antagonist is an anti-EGFR antibody fragment. In some embodiments, the EGFR antagonist is a human or humanized anti-EGFR monoclonal antibody. In some embodiments, the EGFR antagonist is a chimeric anti-EGFR monoclonal antibody. In some embodiments, the EGFR antagonist is selected from cetuximab (e.g., ERBITUX®) necitumumab (IMC-11F8), panitumumab (e.g. VECTIBIX®), and zalutumumab. In some embodiments, the anti-EGFR antibody induces ADCC activity, such as cetuximab, necitumumab, and zalutumumab.

Doses and administration regimens for EGFR antibodies are generally consistent with those provided for their use when used as single agents. For example, in some embodiments, the dose for cetuximab is about 200-600 mg/m². This includes about 200 mg/m², about 250 mg/m², about 300 mg/m², about 350 mg/m², about 400 mg/m², about 450 mg/m², about 500 mg/m², about 600 mg/m², and other doses in that range. Preferably, the cetuximab is administered by intravenous infusion (e.g., as a 30 min., 45 min., 60 min., 90 min, or 120 min infusion). For example, the dose of the cetuximab may be 400 mg/m² administered as a 120-minute intravenous infusion (e.g., maximum infusion rate 10 mg/min), or 200 mg/m² administered as a 60-minute intravenous infusion. For example, in some embodiments, 2 mg/kg cetuximab is administered as an intravenous infusion over 30 minutes every 3 weeks.

In some embodiments, cetuximab may be administered with a higher initial dose followed by lower subsequent doses. The frequency of administration is preferably one time weekly. For example, following an initial dose of cetuximab at 400 mg/m² administered as a 120-minute intravenous infusion (e.g., maximum infusion rate 10 mg/min), subsequent weekly doses may be at 250 mg/m² infused over 60 minutes (e.g., maximum infusion rate 10 mg/min) until disease progression or unacceptable toxicity.

As another example, in some embodiments, the dose for panitumumab is about 1-10 mg/kg. This includes about 1 mg/kg, about 2 mg/kg, about 3 mg/kg, about 4 mg/kg, about 5 mg/kg, about 6 mg/kg, about 7 mg/kg, about 8 mg/kg, about 10 mg/kg and other doses in that range. Panitumumab is administered by intravenous infusion (e.g., as a 30 min., 45 min., 60 min., 90 min, or 120 min infusion). For example, the dose of panitumumab may be 4 mg/kg administered as a 30-, 60-, or 90-minute intravenous infusion. Doses higher than 1000 mg should be administered over 90 minutes. The frequency of administration may be one time every 7 to 21 days (e.g., once every 10, 14, 18, etc. days).

In some embodiments, panitumumab may be administered with a higher initial dose followed by lower subsequent doses or maintenance dose. For example, following an initial dose of panitumumab at 6 mg/kg administered as a 90-minute intravenous infusion, subsequent weekly doses may be at 2-4 mg/kg infused over 30 minutes until disease progression or unacceptable toxicity.

In some embodiments, a combination of the disclosure, optionally including an EGFR antagonist such as cetuximab, is used for the treatment of locally or regionally advanced squamous cell carcinoma of the head and neck. In some embodiments, a combination of the disclosure, optionally including an EGFR antagonist such as cetuximab, is used for the treatment of recurrent or metastatic squamous cell carcinoma of the head and neck progressing after platinum-based therapy. The disclosure also provides methods of increasing the effectiveness of cetuximab in the treatment of head and neck cancer through use in a combination therapy that further comprises a TLR8 agonist and a PD-1 antagonist.

As another example, in one aspect, the combination therapy of the disclosure is amenable for use in the treatment of metastatic colorectal cancer. Colorectal cancer includes the well-accepted medical definition that defines colorectal cancer as a medical condition characterized by cancer of cells of the intestinal tract below the small intestine (i.e. the large intestine (colon), including the cecum, ascending colon, transverse colon, descending colon, and sigmoid colon, and rectum). In some embodiments, such a combination is used for the treatment of EGFR-expressing metastatic colorectal carcinoma. In some embodiments, such a combination is used for the treatment of EGFR-expressing metastatic colorectal carcinoma in patients who are refractory to irinotecan-based chemotherapy. In some embodiments, an EGFR antagonist, such as panitumumab, is administered as part of the combination therapy for the treatment metastatic colorectal carcinoma with disease progression on or following fluoropyrimidine, oxaliplatin, and irinotecan chemotherapy regimens. The disclosure also provides methods of increasing the effectiveness of cetuximab in the treatment of metastatic colorectal carcinoma through use in a combination therapy that further comprises a TLR8 agonist and a PD-1 antagonist.

In some embodiments, it may be preferred to identify whether the cancer is resistant to EGFR antagonism, such as cancers in which resistance to cetuximab is mediated by upregulation of protein production or overexpression of HER2/neu protein. In cases in which such resistance is observed, further treatment with HER2/neu-targeting medicaments such as trastuzumab or lapatinib may be indicated.

Suitable therapeutic agents that can be used in combinations described herein are described in Remington: The Science and Practice of Pharmacy, 22nd Ed. (Pharmaceutical Press 2012), and in Goodman and Gilman's the Pharmacological Basis of Therapeutics, 12th Ed. (McGraw Hill Education 2011). Other suitable therapeutic agents are known to those of skill in the art.

The combinations and methods of the disclosure employ amount of the constituent active agents that are effective in combination for the intended use. Particular dosages of TLR8 agonists, PD-antagonists, and therapeutic agents are also selected based on a number of other factors including the age, sex, species and/or condition of the patient. Effective amounts can also be extrapolated from dose-response curves derived from in vitro test systems or from animal models. The selection of doses for a particular patient, or in the context of a particular cancer type, is within the skill of the practicing physician.

In certain embodiments, the TLR8 agonist is administered prior to, concurrently with, or subsequent to the administration of the one or more therapeutic agents. In one embodiment, the TLR8 agonist is formulated with one or more therapeutic agents. In another embodiment, the one or more therapeutic agents is administered in a separate pharmaceutical composition. In accordance with this embodiment, the one or more therapeutic agents may be administered to a subject by the same or different routes of administration as those used to administer the TLR8 agonist.

### Solid Tumors

The type of cancer that is treated by the methods of the present disclosure can be a solid cancer, such as ovarian cancer, breast cancer, head and neck cancer, renal cancer, bladder cancer, hepatocellular cancer, colorectal cancer, and the like. In some embodiments, the cancer is squamous cell carcinoma of the head and neck (SCCHN) or recurrent or persistent platinum-resistant epithelial ovarian, fallopian tube, or primary peritoneal carcinoma. Other types of cancers that can be treated by the methods of the present disclosure include, but are not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Merkel cell carcinoma, Ewing's tumor, leiomyosarcoma, rhabdomyosarcoma, colon carcinoma, pancreatic cancer, prostate cancer, squamous cell carcinoma, basal cell carcinoma, adenocarcinoma, adenocarcinoma unknown primary sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, gastric, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

According to some embodiments, methods are provided for controlling solid tumor growth (e.g., head and neck, breast, prostate, melanoma, ovarian, renal, colon, cervical tumor growth) and/or metastasis comprising administering an effective amount of a combination of the disclosure to a subject in need thereof. In some embodiments, the subject is a mammal. In some embodiments, the mammal is human.

The term “tumor” is used to denote neoplastic growth which may be benign (e.g., a tumor which does not form metastases and destroy adjacent normal tissue) or malignant/cancer (e.g., a tumor that invades surrounding tissues, and is usually capable of producing metastases, may recur after attempted removal, and is likely to cause death of the host unless adequately treated). As used herein, the terms “tumor”, “tumor growth,” and “tumor tissue” can be used interchangeably, and refer to an abnormal growth of tissue resulting from uncontrolled progressive multiplication of cells and serving no physiological function.

In some embodiments, combination therapy of the disclosure is amenable for use in the treatment of head and neck cancer. The head and neck section is an assembly of a plurality of organs, and the primary foci of head and neck cancer include the paranasal sinus, the epipharynx, the oropharynx, the oral cavity, the hypopharynx, the larynx, and the salivary glands. Head and neck cancer includes cancers of the head or neck region of the body. Most head and neck cancers are squamous cell carcinomas, but some may be exophilic or endophilic. Examples of head and neck cancers include but are not limited to the lip, oral cavity (mouth), tongue, throat, trachea, nasal cavity, paranasal sinuses, pharynx, larynx, thyroid, salivary glands and cervical lymph nodes of the neck, and the like.

### Hematological Cancers

The present disclosure provides methods of treating or ameliorating hematological cancers comprising administering an effective amount of a combination therapy of the disclosure to a subject in need thereof.

Hematological cancers are the type of cancer that affects blood, bone marrow, or lymph nodes. As the three are intimately connected through the immune system, a disease affecting one of the three will often affect the others as well. Hematological cancers may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes, and myeloproliferative diseases are myeloid in origin.

Hematological cancers that may be treated or ameliorated using combinations of the present disclosure include, but are not limited to, lymphomas, leukemias, and myelomas. For example, hematological cancers include Hodgkin's disease, non-Hodgkin's lymphoma (e.g., small lymphocytic lymphoma, follicular center cell lymphoma, lymphoplasmacytoid lymphoma, marginal zone lymphoma, mantle cell lymphoma, immunoblastic lymphoma, Burkitt's lymphoma, lymphoblastic lymphoma, peripheral T-cell lymphoma, anaplastic large cell lymphoma and intestinal T-cell lymphoma), acute lymphocytic leukemia, acute myelocytic leukemia (myeloblastic, promyelocytic, myelomonocytic, monocytic and erythroleukemic); chronic leukemia (chronic myelocytic (granulocytic) leukemia and chronic lymphocytic leukemia, and plasma cell neoplasms including multiple myeloma; polycythemia vera, Waldenstrom's macroglobulinemia, and heavy chain disease.

In some embodiments, the combinations of the disclosure are used to treat a cancer in patients who have had prior platinum-based therapy. In some embodiments, the combinations of the disclosure are used to treat patients having a cancer that is resistant to prior platinum-based therapy. In some embodiments, the combinations of the disclosure are used to treat patients having a cancer that is refractory to prior platinum-based therapy.

In some embodiments, the combinations of the disclosure are used to treat cancer in patients in whom the cancer is recurrent and/or metastatic.

In the methods of the disclosure, it is anticipated that patient benefit may be observed in various parameters. For example, patient benefit may be seen in prolonged median progression-free survival time. Alternatively, patient benefit may be seen in increased median overall survival.

In some embodiments, the disclosure relates to a pharmaceutical composition for treating a cancer in a patient, comprising motolimod in an amount suitable for administering a dose of about 0.1 mg/m² to about 10 mg/m², or about 0.5 mg/m² to about 5 mg/m², of motolimod to the patient, and the composition is co-administered in combination with a PD-1 antagonist in a dose of about 5 mg/m² to about 200 mg/m². In some embodiments, the disclosure relates to a pharmaceutical composition for treating a cancer in a patient, comprising motolimod in an amount suitable for administering a dose of about 0.1 mg/m² to about 10 mg/m², or about 0.5 mg/m² to about 5 mg/m², of motolimod to the patient, and the composition is co-administered in combination with an anti-PD-1 antibody or anti-PDL1 antibody in a dose of about 100 mg/m² to about 500 mg/m². In some embodiments, the composition is also co-administered in combination with an anthracycline compound in a dose of about 25 mg/m² to about 100 mg/m². In some embodiments, the composition is also co-administered in combination with an EGFR antagonist, such as anti-EGFR antibody.

To illustrate, in some embodiments, the disclosure relates to a pharmaceutical composition for treating a cancer, such as ovarian cancer in a patient, comprising motolimod in an amount suitable for administering a dose of about 0.1 mg/m² to about 10 mg/m² of motolimod to the patient, and the composition is co-administered in combination with an anti-PD-1 antibody in a dose of about 1 mg/kg to about 10 mg/kg, optionally also co-administered in combination with an anthracycline compound in a dose of about 40 mg/m² to about 60 mg/m².

As a further illustration, in some embodiments, the disclosure relates to a pharmaceutical composition for treating a cancer, such as head and neck cancer in a patient, comprising motolimod in an amount suitable for administering a dose of about 0.1 mg/m² to about 10 mg/m², or about 0.5 mg/m² to about 5 mg/m², of motolimod to the patient, and the composition is co-administered in combination with an anti-PD-1 antibody in a dose of about 1 mg/kg to about 10 mg/kg, optionally also co-administered in combination with an anti-EGFR antibody, such as cetuximab in a dose of about 100 mg/m² to about 500 mg/m², or panitumumab in a dose of about 1 mg/kg to about 10 mg/kg.

In some embodiments, the disclosure relates to a use of motolimod in the manufacture of a medicament for treatment of cancer in combination with a PD-1 antagonist, wherein the medicament comprises 1 to 150 mg of motolimod, and wherein the medicament is suitable for administration to a human and the medicament is co-administered with a PD-1 antagonist and optionally a therapeutic agent (e.g., an anthracycline compound or an EGFR antagonist).

In some embodiments, the disclosure relates to a pharmaceutical composition for treatment of a cancer in a patient comprising motolimod as an effective ingredient, wherein the composition is co-administered in combination with a PD-1 antagonist, optionally in combination with a therapeutic agent (e.g., an anthracycline compound or an EGFR antagonist).

In some embodiments, the disclosure relates to a compound for use in a method of treating cancer, wherein the compound is motolimod, and wherein the method comprises administering the compound at a dose of about 0.1 mg/m² to about 150 mg/m² in combination with a dose of about 1 mg/kg to about 10 mg/kg of a PD-1 antagonist, wherein the compound is administered concurrently with the PD-1 antagonist and the cancer is head and neck cancer, ovarian cancer, or other cancer. In various embodiments, depending on the cancer to be treated and other factors, the compound is administered in further combination with a therapeutic agent, such as an anthracycline compound or an EGFR antagonist.

Over the course of treatment, therapeutically effective combinations can be adjusted (e.g., personalized) to a particular patient based upon how the patient is responding to the treatment. More specifically, the relative amounts and timing of administration of one or more components (i.e., a PD-1 antagonist, a TLR8 agonist, or a therapeutic agent) of a therapeutically effective combination can be adjusted (i.e., increased or decreased) depending on the patient's response to a prior therapeutically effective combination. For example, if the treatment alters the frequency of or function of a population of immune-related cells, thereby lessening the patient's ability to fight a cancer, the therapeutically effective combination can be adjusted to undo this alteration and promote a positive clinical outcome.

A non-limiting example of such an alteration is an increase in a population of immune-related cells known as the regulatory T cells (Treg). An increase in Treg frequency is associated with suppressive effects on antibody-dependent cellular cytotoxicity (ADCC). Elevated Treg frequency can result in a poorer clinical outcome in cancer therapies, including, for example, in cases where ADCC would be implicated. Certain therapeutic agents (e.g., cetuximab) have been shown to increase Treg frequencies; if a patient has received a therapeutically effective combination of the disclosure that comprises such an therapeutic agent and has had an increase in Treg frequency, the relative amount of that therapeutic agent in a subsequent therapeutically effective combination can be reduced to undo the patient's increase in Treg frequency. Thus, in a second therapeutically effective combination, the amount of the therapeutic agent that likely increases Treg frequency can be decreased and/or the amount of one or more other active agents can be increased. If subsequent to treatment with the second therapeutically effective combination, the patient's Treg frequency is further altered (either continues to increase or decreases), then his/her third therapeutically effective combination may be further adjusted as necessary to enhance the patient's ADCC and to promote a positive clinical outcome. This pattern of detecting the frequency of Treg cells and adjusting a subsequent therapeutically effective combination can be repeated until completion of treatment.

The frequency of Tregs in a biological sample from a patient can be determined by any method known in the art. Such methods may include quantifying biomarkers associated with Tregs. Examples of such biomarkers include, but are not limited to, CD4, CD25, CD39, CTLA-4 (CD152), FoxP3, and Lap. The biological sample can be a blood sample or a sample obtained from a tumor biopsy. The biomarkers can be quantified by any method known in the art, e.g., RT-PCR, microarray, in situ hybridization, and an antibody-mediated assay.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

The term “administration”, “administering”, “co-administration”, or “co-administering” refers to both concurrent and sequential administration of the component agents of the combination therapy.

A “subject” or “patient” in the context of the present disclosure is preferably a mammal. The mammal can be a human, non-human primate, mouse, rat, dog, cat, horse, or cow, but are not limited to these examples. A subject can be male or female.

“ADCC activity” as used herein refers to an activity to damage a target cell (e.g., tumor cell) by activating an effector cell via the binding of the Fc region of an antibody to an Fc receptor existing on the surface of an effector cell such as a killer cell, a natural killer cell, an activated macrophage or the like. An activity of antibodies of the present disclosure includes ADCC activity. ADCC activity measurements and antitumor experiments can be carried out in accordance using any assay known in the art.

The term “enhances antibody-dependent cellular cytotoxicity”, “enhances ADCC” (e.g., referring to cells), or “increasing ADCC” includes any measurable increase in cell lysis when contacted with a combination comprising a therapeutic antibody as compared to the cell killing of the same cell in contact with the therapeutic antibody alone. For example, an increase in cell lysis may be by at least about 10%, 20%, 30%, 40%, 50%, 60%, 70%, 80%, 90%, 100%, 150%, 200%, 250%, 300%, 325%, 400%, or 500%.

The term “monoclonal antibody” or “monoclonal antibody composition” as used herein means a preparation of antibody molecules of single molecular composition. A monoclonal antibody composition displays a single binding specificity and affinity for a particular epitope.

The term “human antibody” as used herein means an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo).

The term “human monoclonal antibody” means an antibody displaying a single binding specificity and having variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell. The term “human monoclonal antibody” as used herein, also means a human antibody that is prepared, expressed, created or isolated by recombinant means, such as an antibody that is (a) isolated from an animal (e.g., a mouse) that is transgenic or transchromosomal for human immunoglobulin genes or a hybridoma prepared therefrom (described further below), (b) isolated from a host cell transformed to express the human antibody, e.g., from a transfectoma, (c) isolated from a recombinant, combinatorial human antibody library, and (d) prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “humanized antibody” is intended to refer to an antibody in which CDR sequences derived from the germline of another mammalian species, such as mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “mutated gene” or “mutation” or “functional mutation” or “mutant” refers to an allelic form of a gene, which is capable of altering the phenotype of a subject having the mutated gene relative to a subject which does not have the mutated gene (i.e., wild-type). The altered phenotype caused by a mutation can be corrected or compensated for by certain agents. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region of a gene.” A specific genetic sequence at a polymorphic region of a gene is an allele. A polymorphic region can be a single nucleotide, the identity of which differs in different alleles. A polymorphic region can also be several nucleotides long.

As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. Thus, for example, a reference to “a host cell” includes a plurality of such host cells, and a reference to “an antibody” is a reference to one or more antibodies and equivalents thereof known to those skilled in the art, and so forth. Further, the use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “effective amount” of refers to an amount sufficient to provide the desired anti-cancer effect, anti-tumor effect or anti-disease effect in an animal, preferably a human, suffering from cancer or a cellular disease. Desired anti-tumor effects include, without limitation, the modulation of tumor growth (e.g. tumor growth delay), tumor size, or metastasis, the reduction of toxicity and side effects associated with a particular anti-cancer agent, the amelioration or minimization of the clinical impairment or symptoms of cancer, extending the survival of the subject beyond that which would otherwise be expected in the absence of such treatment, and the prevention of tumor growth in an animal lacking any tumor formation prior to administration, i.e., prophylactic administration.

The term “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

It is understood that modifications which do not substantially affect the activity of the various embodiments of this invention are also provided within the definition of the invention provided herein. Accordingly, the following examples are intended to illustrate but not limit the present invention. While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

### EXAMPLE 1

### Motolimod Enhancement of Cetuximab-Mediated ADCC in Head and Neck Cancer

Cytotoxic activity of PBMC or isolated NK was determined using a ⁵¹Cr release assay during which PBMC pre-treated with TLR8 agonist motolimod (250 nM 18 hr) were incubated with EGFR^{|} UM-22B HNC cells and cetuximab (10 μg/mL) for 4 hr. PBMC cytokine release was also measured using a multiplex cytokine assay. FcγR III-158 genotype was determined using a quantitative PCR-based assay kit. Significant differences in ADCC by FcγR Ma or TLR8 genotype were determined using a Kruskal-Wallis one-way analysis of variance, and a post hoc Mann-Whitney non-parametric t test was performed for differences between groups. For cytokine analysis, a Mann-Whitney non-parametric test was performed.

TLR8 stimulation enhanced ADCC lytic activity in NK expressing FcγR IIIc FF genotype (2.6-fold), VF (1.7-fold) and VV (1.8-fold), with significant difference between genotypes (p=0.0389). Enhancement was significantly greater in FcγR IIIc FF genotype than NK with a V allele (p=0.0130). NK cells were the primary effectors in motolimod-enhanced, cetuximab-mediated ADCC. Th1 cytokines were secreted at significantly higher levels in VTX 2337-treated PBMC versus untreated PBMC: TNF-α (p<0.0001), IFN-γ (p<0.0001), IL-12p40 (p=0.0002), and IL-12p70 (p=0.03). TLR8 stimulated PBMC from HNC patients were more avid effectors in cetuximab-mediated ADCC (p=0.0262), and this treatment caused an additional subset of HNC patient PBMC to manifest cetuximab-mediated ADCC.

Motolimod enhanced cetuximab-mediated ADCC against HNC cells, and induced cetuximab-mediated lytic activity in PBMC from HNC patients that did not initially mediate ADCC. Three key Th1 cytokines (TNF-α, IFN-γ and IL-12) were preferentially stimulated by motolimod-treated PBMC.

### EXAMPLE 2

### Clinical Data: Single Agent Cetuximab

In a phase 2 study of HNC patients treated with a novel neoadjuvant single-agent cetuximab, immunosuppressive phenotype and activation status of Treg and NK cells were analyzed in the circulation and tumor microenvironment. Cetuximab treatment increased the frequency of CD4⁺FOXP3⁺ intratumoral Treg expressing CTLA-4, CD39 and TGF-β. These Treg suppressed cetuximab-mediated ADCC and their presence correlated with poor clinical outcome in two prospective clinical trial cohorts. Cetuximab expanded CTLA-4⁺FOXP3⁺ Treg in vitro, in part by inducing DC maturation, in combination with TGF-β and T cell receptor triggering. Importantly, cetuximab-activated NK cells selectively eliminated intratumoral Treg but preserved effector T cells. See, Jie et al., Cancer Res, June 1; 75(11):2200-10, (2015)_[Epub 2015 Apr. 1].

### EXAMPLE 3

### Clinical Data: Motolimod with Cetuximab

A phase 1b study was conducted, evaluating subjects with recurrent or metastatic SCCHN. Motolimod was administered on days 1, 8, and 15, with weekly cetuximab in 28-day cycles, with escalating doses of motolimod and fixed doses of cetuximab using a 3+3 design. The study determined that motolimod can be administered in combination with cetuximab with an acceptable safety profile and with pharmacologic evidence of dose-dependent clinical activity. See, Chow et al., Int J Radiat Oncol Biol Phys., 2014, 88(2):503-504.

### EXAMPLE 4

### Preclinical Data: Motolimod with Cetuximab

In vitro studies have demonstrated that motolimod significantly enhances cetuximab-mediated ADCC and DC IVS of CD8⁺ T cells in HNC. The primary effectors for the increase in cytotoxicity are NK cells. In addition, TLR8 stimulation by motolimod in combination with cetuximab enhances DC maturation and cross-priming of CD8⁺ cells. See, Stephenson et al., Cancer Immunol Immunother, 2013 62(8):1347-57.

### EXAMPLE 5

### Clinical Trial Design: Motolimod+Cetuximab+Nivolumab

A clinical study may be performed to confirm the utility of a method of treatment of head and neck cancer using a combination of a TLR8 agonist, a PD-1 antagonist, and an EGFR antagonist. Other study designs are possible.

The primary objective of the study is to confirm the extent to which the administration of neoadjuvant cetuximab and immunotherapy (motolimod or motolimod+nivolumab) modulates immune biomarkers in peripheral blood and squamous cell head and neck cancer (SCCHN) tumors. A secondary, exploratory objective is to assess whether modulation of biomarkers can predict anti-tumor response.

The primary endpoint of the study is assessment of the change in immune biomarkers following 3-4 weeks of neoadjuvant cetuximab and immunotherapy (motolimod or motolimod+nivolumab). A secondary endpoint is measurement of modulation of induction of inflammatory markers.

Inclusion criteria for patients to be included in the trial are any of the following: patients having histologically or cytologically confirmed SCCHN; previously untreated stage II, III, or IVA disease; primary tumors of the oral cavity, oropharynx, hypopharynx, or larynx; planned macroscopic complete resection of the primary tumor; and ECOG performance status of 0 or 1.

Exclusion criteria for exclusion from the trial include any of the following: patients having primary tumors of the sinuses, paranasal sinuses, or nasopharynx, or unknown primary tumor or evidence of distant metastasis, or any other malignancy active with 5 years except for non-melanoma skin cancer or carcinoma in situ of the cervix, DCIS or LCIS of the breast; patients with a previous history of HNC and patients with active autoimmune disease requiring therapy.

To assess responses of subjects, the subjects are evaluated by CT or MRI scan, and blood is drawn and tumor tissue biopsied prior to and after the 3-4 week preoperative treatment.

Correlative studies include, for example, pharmacogenomics, such as genetic polymorphisms that may impact the response to TLR8 agonist or cetuximab; immune biomarker analysis, such as cytokines, chemokines, and inflammatory markers in serum; TLR8 biomarker response; cellular immune response such as CD3, CD4, CD8, CD14, CD11, HLA-DR, CD19; and TCR sequencing.

Up to twenty-seven (n=27) patients with complete specimens (tumor, peripheral blood mononuclear cells (PBMC) and serum) are enrolled in the study in 2 cohorts:

Cohort 1 (n=12-15): Treatment with motolimod and cetuximab;

Cohort 2 (n=12): Treatment with motolimod, cetuximab, and nivolumab.

The dose of motolimod is 2.5 mg/m² or 3.0 mg/m², administered by subcutaneous injection on Days 1, 8 and 15.

The initial dose of cetuximab is 400 mg/m² intravenously administered over 120 minutes on Day 1, followed by weekly infusions at 250 mg/m² IV over 60 minutes on Days 8 and 15.

The dose of nivolumab is 1.5 mg/kg or 3.0 mg/kg, administered by intravenous infusion over 60 minutes on Days −7 to −1, and on Day 15.

Motolimod is administered prior to cetuximab. In Cohort 2, nivolumab is administered last on Day 15.

Patients are then progressed to surgery for resection of the tumor within 2 days of the last dose of motolimod.

Cohort 2A (n=6): nivolumab start dose (1.5 mg/kg). If there are no significant and/or unacceptable toxicities related to the neoadjuvant therapy (including delay of surgery for more than 3 days after completion of last dose of neoadjuvant therapy for reasons of excessive toxicity related to the neoadjuvant therapy) identified in the first 6 patients, then nivolumab dose may be increased for Cohort 2B. If there are significant and/or unacceptable toxicities identified in the first 6 patients, the cohort is closed.

Cohort 2B (n=6): The nivolumab dose is 3 mg/kg for the 6 patients in this cohort. If there are significant and/or inacceptable toxicities (including delay of surgery for more than 3 days after completion of last dose of neoadjuvant therapy for reasons of excessive toxicity related to the neoadjuvant therapy) identified in at least 2 of the 6 patients, the remaining subjects in cohort 2B are treated at 1.5 mg/kg.

In both cohorts, depending on the surgical schedule, the neoadjuvant therapy are taken for a minimum of 3 weekly doses of motolimod plus cetuximab. The therapy may be extended to a maximum of 4 weeks of motolimod plus cetuximab treatment.

In Cohort 2, in addition to the motolimod plus cetuximab treatment described above, 2 doses of nivolumab are administered. The first dose occurs at least 1 day and no more than 7 days prior to the first dose of cetuximab/motolimod, i.e., during the period of Day −7 to Day −1; the second dose occurs on Day 15.

The nature of complete resection of the primary tumor and neck dissection, including the extent of primary resection, type of reconstruction, and levels of nodes to be dissected, are determined by the treating head and neck surgeon.

### EXAMPLE 6

### Non-Small Cell Lung Cancer Sample Protocol

To illustrate another implementation of the method of the disclosure, in Stage IIIB/IV non-small cell lung cancer, a first phase of treatment can comprise 2.0-3.0 mg/m² motolimod and 10 mg/kg anti-PD-1 antibody, followed by 10 mg/kg anti-PD-1 antibody on day 1 and 2.5 mg/m² motolimod on days 1 and 8 of the first four 21-day cycles, followed by 10 mg/kg anti-PD-1 antibody on day 1 and 2.5 mg/m² motolimod on day 1 of subsequent cycles. Cycles are continued until disease progression.

### EXAMPLE 7

### Non-Small Cell Lung Cancer Sample Protocol

To illustrate another implementation of the method of the disclosure, in the second line treatment of urothelial carcinoma (bladder cancer) following chemotherapy induction, 2.5 mg/m² motolimod is administered on days 1 and 15 of a 28-day cycle, and 10 mg/kg anti-PD-1 antibody is administered on days 1 and 15 of the cycle. Cycles are continued until disease progression.

1. A method for treating cancer comprising administering to a subject in need thereof a combinational therapy comprising a Toll-like receptor-8 (TLR8) agonist and a pharmaceutically acceptable carrier, wherein the combinational therapy further comprises an effective amount of a Programed Death-1 (PD-1) antagonist.

2. The method of claim 1, in which the TLR8 agonist is a compound of Formula I: 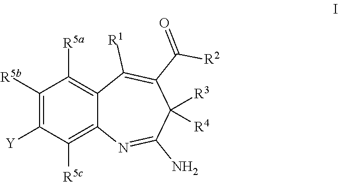
in which,
Y is CF₂CF₃, CF₂CF₂R⁶, or an aryl or heteroaryl ring, wherein said aryl and heteroaryl rings are substituted with one or more groups independently selected from alkenyl, alkynyl, Br, CN, OH, NR⁶R⁷, C(═O)R⁸, NR⁶SO₂R⁷, (C₁-C₆ alkyl)amino, R⁶OC(═O)CH═CH₂—, SR⁶ and SO₂R⁶, and wherein said aryl and heteroaryl rings are optionally further substituted with one or more groups independently selected from F, Cl, CF₃, CF₃O—, HCF₂O—, alkyl, heteroalkyl, and ArO—;
R¹, R³ and R⁴ are independently selected from H, C₁-C₆ alkyl, C₂-C₆ alkenyl, C₂-C₆ alkynyl, C₁-C₆ heteroalkyl, C₃-C₆ cycloalkyl, C₃-C₆ cycloalkenyl, heterocycloalkyl with 3 to 8 ring atoms wherein one atom is selected from nitrogen, oxygen and sulfur, aryl and 5-7 membered heteroaryl, wherein said alkyl, alkenyl, alkynyl, heteroalkyl, cycloalkyl, cycloalkenyl, heterocycloalkyl, aryl and heteroaryl are optionally substituted with one or more substituents independently selected from C₁-C₆ alkyl, C₂-C₆ alkenyl, C₂-C₆ alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH—, NR⁶SO₂R⁷, SR⁶, and SO₂R⁶;
or R³ and R⁴ together with the atom to which they are attached form a saturated or partially unsaturated C₃-C₆ carbocyclic ring, wherein said carbocyclic ring is optionally substituted with one or more substituents independently selected from C₁-C₆ alkyl, C₂-C₆ alkenyl, C₂-C₆ alkynyl, F, Cl, Br, I, CN, OR⁶, NR⁶R⁷, C(═O)R⁶, C(═O)OR⁶, OC(═O)R⁶, C(═O) NR⁶R⁷, (C₁-C₆ alkyl)amino, CH₃OCH₂O—, R⁶OC(═O)CH═CH—, NR⁶SO₂R⁷, SR⁶, and SO₂R⁶;
R² and R⁸ are independently selected from H, OR⁶, NR⁶R⁷, C₁-C₆ alkyl, C₂-C₆ alkenyl, C₂-C₆ alkynyl, C₁-C₆ heteroalkyl, C₃-C₆ cycloalkyl, C₃-C₆ cycloalkenyl, heterocycloalkyl with 3 to 8 ring atoms wherein one atom is selected from nitrogen, oxygen and sulfur, aryl and 5-7 membered heteroaryl, wherein said alkyl, alkenyl, alkynyl, heteroalkyl, cycloalkyl, cycloalkenyl, heterocycloalkyl, aryl and heteroaryl are optionally substituted with one or more substituents independently selected from a C₁-C₆ alkyl, C₂-C₆ alkenyl, C₂-C₆ alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH—, NR⁶SO₂R⁷, SR⁶, and SO₂R⁶;
R⁵a, R⁵b, and R⁵c are independently selected from H, F, Cl, Br, I, OMe, CH₃, CH₂F, CHF₂ and CF₃ and
R⁶ and R⁷ are independently selected from H, C₁-C₆ alkyl, C₂-C₆ alkenyl, C₂-C₆ alkynyl, C₁-C₆ heteroalkyl, C₃-C₆ cycloalkyl, C₃-C₆ cycloalkenyl, heterocycloalkyl with 3 to 8 ring atoms wherein one atom is selected from nitrogen, oxygen and sulfur, aryl and 5-7 membered heteroaryl, wherein said alkyl, alkenyl, alkynyl, heteroalkyl, cycloalkyl, cycloalkenyl, heterocycloalkyl, aryl and heteroaryl are optionally substituted with one or more substituents independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH₂—, NR⁶SO₂R⁷, SR⁶, and SO₂R⁶,
or R⁶ and R⁷ together with the atom to which they are attached form a saturated or partially unsaturated heterocyclic ring, wherein said heterocyclic ring is optionally substituted with one or more groups independently selected from alkyl, alkenyl, alkynyl, F, Cl, Br, I, CN, OR⁶, NR⁶R⁷, C(═O)R⁶, C(═O)OR⁶, OC(═O)R⁶, C(═O)NR⁶R⁷, (C₁-C₆ alkyl)amino, CH₃OCH₂O—, R⁶OC(═O)CH═CH—, NR⁶SO₂R⁷, SR⁶, and SO₂R⁶,
and metabolites, solvates, tautomers, and pharmaceutically acceptable salts thereof.

3. The method of claim 1, in which the TLR8 agonist is motolimod.

4. The method of claim 1, in which the PD-1 antagonist interferes with binding of PD-1 to PD-L1 or PD-L2.

5. The method of claim 1, in which the PD-1 antagonist interferes with a biological activity of PD-1.

6. The method of claim 1, in which the PD-1 antagonist is an antibody that binds to PD-1.

7. The method of claim 6, in which the PD-1 antagonist is selected from nivolumab, pembrolizumab, and pidilizumab.

8. The method of claim 1, in which the PD-1 antagonist is an antibody that binds to PD-L1.

9. The method of claim 8, in which the PD-1 antagonist is selected from atezolizumab, avelumab, BMS-936559, and durvalumab.

10. The method of claim 1, in which the combinational therapy further comprises an epidermal growth factor receptor (EGFR) antagonist.

11. The method of claim 10, in which the EGFR antagonist is cetuximab, necitumumab, panitumumab, or zalutumumab.

12. (canceled)

13. (canceled)

14. The method of claim 1, wherein the combinational therapy further comprises an effective amount of an anthracycline anticancer agent.

15. The method of claim 14, in which the anthracycline anticancer agent is pegylated liposomal doxorubicin.

16. The method of claim 10, wherein the TLR8 agonist is motolimod, the PD-1 antagonist is nivolumab, and the EGFR antagonist is cetuximab.

17. The method of claim 14, wherein the TLR8 agonist is motolimod, the PD-1 antagonist is nivolumab, and the anthracycline anticancer agent is pegylated doxorubicin.

18. A method for improving a combinational therapy of treating cancer, the combinational therapy comprising an anti-EGFR antibody, and a TLR8 agonist, wherein the method consists of administration of a PD-1 antagonist in an amount sufficient to inhibit PD-1 activity.

19. The method of claim 1, wherein the subject is a human.

20. The method of claim 1, wherein the cancer is selected from the group consisting of ovarian cancer, breast cancer, head and neck cancer, renal cancer, bladder cancer, hepatocellular cancer, and colorectal cancer.
